(19) United States
(12) Patent Application Publication
(10) Pub. No.: US 2023/0233429 A1
(43) Pub. Date: Jul. 27, 2023

(54) TEMPORARY HAIR DYE COMPOSITION

(71) Applicant: TOKIWA CORPORATION, Nakatsugawa-shi (JP)

(72) Inventor: Shunpei TAKEZAWA, Kawaguchi-shi (JP)

(21) Appl. No.: 18/155,770

(22) Filed: Jan. 18, 2023

(30) Foreign Application Priority Data
Jan. 25, 2022 (JP) 2022-009643

(51) Int. Cl. A61K 8/36; A61K 8/02; A61K 8/34; A61K 8/25; A61K 8/04; A61Q 5/06

(52) U.S. Cl. CPC A61K 8/361; A61K 8/022; A61K 8/345; A61K 8/25; A61K 8/046; A61Q 5/065; A61K 2800/4322; A61K 2800/882; A61K 2800/624

(57) ABSTRACT

A temporary hair dye composition contains a soap base and a pigment. A method for temporarily coloring hair comprises mixing the temporary hair dye composition including the soap base and the pigment with water to generate a foam, and then applying the foam to the hair.

### CROSS-REFERENCE TO RELATED APPLICATIONS

This application is based upon and claims the benefit of priority from Japanese Patent Application No. 2022-009643, filed on Jan. 25, 2022, the entire contents of which are incorporated herein by reference.

### TECHNICAL FIELD

The present disclosure relates to a temporary hair dye composition.

### BACKGROUND

In recent years, hair dyes have become popular due to an increase in the customer demand for gray hair dyeing resulting from the aging population and the boom in fashionable colored hair. Hair dyes are classified into temporary hair dyes, semi-permanent hair dyes, and permanent hair dyes based on the durability of color applied to the hair. Among these, the temporary hair dyes have advantages that their application is simple and the hair can be quickly dyed by simply applying the dye to a desired part, and thus they are suitable for use before going out. In addition, since the temporary hair dyes can be easily washed off with shampoo, they are convenient when people are dissatisfied with a dyed color or want to change it to a different color, and their demand is high in the market.

In the related art, a spray product, which may be referred to as a temporary hair dye, is obtained by filling a composition containing a coloring material mainly composed of an inorganic pigment, a coating agent (for fixing the coloring material to the hair and preventing secondary adhesion to clothes) and a quick-drying volatile solvent such as ethanol into an aerosol can together with an aerosol propellant. For example, refer to Japanese Unexamined Patent Publication No. 2004-189634.

In the above temporary hair dye, the coating agent employs a polymer compound having a coating property, which tends to make the colored hair coarse. In order to reduce the feeling of coarse hair, a temporary hair dye containing a gelling agent and an oily substance has also been proposed. For example, refer to Japanese Unexamined Patent Publication No. H10-158129.

### SUMMARY

However, the technology in the related art faced a difficulty in sufficiently preventing secondary adhesion of the coloring material.

Disclosed herein is a temporary hair dye composition that is configured to allow hair to be colored with finishing of less hair coarseness as well as to be capable of sufficiently preventing secondary adhesion.

In some examples, the temporary hair dye composition comprises (A) a soap base and (B) a pigment.

With the aforementioned configuration, the temporary hair dye composition can color the hair with the finishing of less hair coarseness and can sufficiently prevent the secondary adhesion. It may be inferred that since the temporary hair dye composition comprises a soap base, a property of fixing a pigment to the hair and a property of reducing friction resistance of the hair to which the pigment is fixed are obtained at the same time, and thus a property of resistance to the secondary adhesion improves efficiently. This rational may account for the obtained effects.

In some examples, the temporary hair dye may have a water content of 20% or less as measured by a Karl Fischer method.

In some examples, the temporary hair dye composition may be in the form of a powder or solid.

In some examples, the content of the component (A) may be 30 to 90 mass % based on the total amount of the temporary hair dye composition.

In some examples, the mass ratio (A)/(B) of the content of the component (A) to the content of the component (B) may be 0.5 to 80.

In some examples, the component (B) may comprise (B1) a color pigment and (B2) an extender pigment. In some examples, the content of the component (B1) may be 0.5 to 50 parts by mass and the content of the component (B2) may be 0.5 to 50 parts by mass, based on the total content of 100 parts by mass of the component (A), the component (B1) and the component (B2).

In some examples, the total content of the component (B1) and the component (B2) may be 1 to 55 parts by mass.

In some examples, the temporary hair dye composition may further comprise a nonionic surfactant.

The temporary hair dye composition can be used by being mixed with water.

### DETAILED DESCRIPTION

Before addressing details of example embodiments described below, some terms are defined or clarified.

As may be used herein, the terms “comprises,” “comprising,” “includes,” “including,” “has,” “having” or any other variation thereof, are intended to cover a non-exclusive inclusion. For example, a composition that comprises a list of elements is not necessarily limited to only those elements but may include other elements not expressly listed or inherent to such composition.

Unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed provided that these additional included materials, steps, features, components, or elements do not materially affect the basic and novel characteristic(s) of the claimed invention. The term ‘consisting essentially of’ occupies a middle ground between “comprising” and “consisting of.”

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Also, use of “a” or “an” is employed to describe elements and components described herein. This is done merely for convenience and to give a general sense of the scope of the invention. This description should be read to include one or at least one and the singular also includes the plural unless it is obvious that it is meant otherwise.

Temporary Hair Dye Composition

An example temporary hair dye composition comprises (A) a soap base (hereinafter also referred to as a component (A)), and (B) a pigment (hereinafter also referred to as a component (B)).

(A) Soap Base

A usable soap base may be a soap base used in cosmetics. For example, there may be used a soap base prepared by saponifying animal fats and oils, such as beef tallow and sheep oil, or vegetable fats and oils, such as coconut oil, palm oil, palm kernel oil, soybean oil, olive oil, cottonseed oil and their mixed ones, with an alkaline aqueous solution such as a caustic soda aqueous solution or a caustic potash aqueous solution, or by neutralizing fatty acids obtained from decomposing these fats and oils with an alkaline aqueous solution such as a caustic soda aqueous solution, a caustic potash aqueous solution, or an alkanolamine aqueous solution. The soap base in a neat soap state or a dried form may be added to the temporary hair dye composition.

The soap base may comprise a fatty acid salt having 10 to 40 carbon atoms and may comprise a plant-derived fatty acid salt such as a palm kernel fatty acid salt, a palm fatty acid salt, a hydrogenated palm fatty acid salt, or a sunflower seed oil fat salt, and may comprise a higher fatty acid salt such as a myristate, stearate, or isostearate. These salts may be alkali salts, such as sodium salts and potassium salts, amino acid salts, such as aspartates and arginine salts, or amine salts such as triethanolamine salts. The soap base may contain either or both of a solid soap and a liquid soap. In addition, the soap base may comprise a pH adjusting agent such as citric acid or sodium hydroxide. In the displayed components of the soap base, the name of a fatty acid salt may be described as being divided into a fatty acid and an alkali that neutralizes it, and the name of a plant-derived fatty acid may be described such that sunflower seed oil fatty acid, for example, is sunflower seed oil.

The soap base may or may not comprise water. The water content in the soap base may be 0.1 to 80 mass %, 0.5 to 20 mass %, or 1.0 to 18 mass %. The water content as used in this specification means a value measured by the Karl Fischer method.

The content of the fatty acid salt in the soap base may be 20 to 100 mass % from the viewpoint of pigment fixation, and may be 60 to 95 mass % or 70 to 90 mass % from the viewpoint of achieving both the pigment fixation and transfer resistance, based on the total amount of the soap base.

The content of the fatty acid salt can be measured according to “JIS K3304: 2019 (6.4 pure soap content).”

The usable soap base may employ a commercial product such as “Palmsabun PS-2405” (product name, IOI OLEOCHEMICAL), “SOAPLEX HOSOVEG” (product name, commercially available from Laboratoire Sandralex), “ALFOAM K-100” (product name, commercially available from New Japan Chemical Co., Ltd.), “ORGANIC PPAQUE SOAP BASE” (product name, commercially available from CARIBBEAN ECO SOAPS), or “Aminosoap AR-12” (product name, commercially available from Ajinomoto Healthy Supply Co., Inc.).

The content of the component (A) in the temporary hair dye composition based on the total amount of the temporary hair dye composition may be 30 to 90 mass %; from the viewpoint of achieving favorable foaming, reduction of hair coarseness, and transfer resistance all at the same time, the content may be 35 to 80 mass % or 40 to 80 mass %; and from the viewpoint of easily obtaining color development, the content may be 40 to 60 mass %.

The amount of the component (A) to be added may be set using the content of the fatty acid salt contained in the component (A) as an index (i.e., in terms of the mass of the fatty acid salt). The amount of the component (A) to be added in terms of the mass of the fatty acid salt may be 20 to 90 mass %, 30 to 80 mass %, or 35 to 75 mass %, based on the total amount of the temporary hair dye composition. Specifically, the content of the fatty acid salt derived from the component (A) in the temporary hair dye composition may be within the above numerical range.

(B) Pigment

Pigments used in cosmetics may be added to the temporary dye composition as the pigment; and color pigments, extender pigments and the like may be exemplified. The pigment powder can be used in any shape such as a spherical shape, a plate shape or a needle shape; in any particle size such as a fume size, a fine particle size, or a pigment grade size; and in any particle structure such as a porous or non-porous structure. The component (B) may be used alone, or two or more thereof may be used in combination.

The temporary hair dye composition may comprises (B1) a color pigment (hereinafter also referred to as a component (B1)) as the component (B). The component (B1) may be used alone, or two or more thereof may be used in combination.

Examples of the color pigment include red oxide, yellow iron oxide, black iron oxide, cobalt oxide, chromium oxide, ultramarine blue, Prussian blue, titanium oxide, zinc oxide, fine particle titanium oxides, pearl pigments (mica titanium, iron oxide coated mica titanium, fine particle titanium oxide coated mica titanium, barium sulfate coated mica titanium, fish scale flakes, bismuth oxychloride, aluminum flakes, glass powder, etc.), organic pigments (red No. 228, red No. 226, cyan No. 404, red No. 202, yellow No. 4 aluminum lake, etc.), and natural pigments (carmine, safflower, etc.). These pigments may be subjected to surface treatment in order to improve usability, dispersibility and the like. For the surface treatment, metal soaps, silicone compounds, fluorine compounds, surfactants, and amino acid compounds other than N-acyl acidic amino acids may be used.

The particle size of the component (B1) may be 1 to 500 μm or 5 to 300 μm. The particle size of the component (B1) may be measured by the following example method.

For example, the particle size is a numerical value obtained by measurement using laser diffraction scattering or observation using an ultra-high voltage transmission electron microscope (TEM) or a scanning electron microscope (SEM), and may be a particle size indicated on a product when a commercially available product is used.

The content of the component (B1) in the temporary hair dye composition may be 0.5 to 50 mass % from the viewpoint of color development, and may be 1.0 to 35 mass %, or 2.0 to 20 mass % from the viewpoint of easily achieving both the color development and transfer resistance, based on the total amount of the temporary hair dye composition.

The temporary hair dye composition may comprise (B2) an extender pigment (hereinafter also referred to as a component (B2)) as the component (B). The component (B2) may be used alone, or two or more thereof may be used in combination.

Examples of the extender pigment include inorganic powders, synthetic inorganic powders, organic powders, metal soaps, and synthetic polymer powders. More specifically, there may be mentioned anhydrous silicic acid, aluminum silicate, magnesium silicate, magnesium aluminum silicate, mica, synthetic phlogopite (synthetic mica), kaolin, sericite, synthetic sericite, talc, phlogopite, synthetic mica, silica, calcium carbonate, magnesium carbonate, aluminum oxide, boron nitride, silicon carbide, barium sulfate, zinc stearate, aluminum stearate, zinc myristate, polyethylene powder, urethane beads, polymethyl methacrylate, and organopolysiloxane elastomers.

The particle size of the component (B2) may be 1 to 200 μm or 5 to 150 μm. The particle size of the component (B2) may be measured by the following example method.

For example, the particle size is a numerical value obtained by measurement using laser diffraction scattering or observation using an ultra-high voltage transmission electron microscope (TEM) or a scanning electron microscope (SEM), and may be a particle size indicated on a product when a commercially available product is used.

The content of the component (B2) in the temporary hair dye composition based on the total amount of the temporary hair dye composition may be 0.5 to 55 mass % from the viewpoint of reducing hair coarseness; the content may be 3 to 50 mass % from the viewpoint of easy foaming; and the content may be 10 to 40 mass % from the viewpoint of achieving both the reduction of hair coarseness and transfer resistance.

The temporary hair dye composition may comprise (B1) a color pigment and (B2) an extender pigment as the component (B). The respective contents of the component (B1) and the component (B2) may be 0.5 to 50 parts by mass and 0.5 to 50 parts by mass, 1.0 to 45 parts by mass and 20 to 50 parts by mass, or 1.0 to 35 parts by mass and 20 to 40 parts by mass, based on the total amount of the temporary hair dye composition.

From the viewpoints of smooth feel during use and transfer resistance, the total content of the component (B1) and the component (B2) may be 1 to 55 parts by mass, 5 to 45 parts by mass, or 10 to 40 parts by mass, based on the total content of 100 parts by mass of the component (A), the component (B1) and the component (B2).

From the viewpoint of achieving color development, reduction of hair coarseness and transfer resistance all at the same time, the respective contents of the component (A), the component (B1) and the component (B2) may be 40 to 60 mass %, 1.5 to 20 mass %, and 10 to 35 mass %, or may be 40 to 60 mass %, 1.5 to 15 mass %, and 20 to 35 mass %, based on the total amount of the temporary hair dye composition.

From the viewpoints of color development and reduction of hair coarseness, the content of the component (B) in the temporary hair dye composition may be 1 to 60 mass %, 5 to 50 mass %, 12 to 44 mass %, 15 to 40 mass %, 20 to 38 mass % by mass, or 25 to 36 mass %, based on the total amount of the temporary hair dye composition. From the viewpoint of transfer resistance, the mass ratio (A)/(B) of the content of the component (A) to the content of the component (B) may be 0.50 or more, 0.60 or more, 0.70 or more, 0.80 or more, 0.90 or more, 1.00 or more, 1.10 or more, 1.20 or more, 1.30 or more, 1.40 or more, 1.50 or more, or 1.55 or more. From the viewpoint of the color development, the mass ratio (A)/(B) of the content of component (A) to the content of component (B) may be 80.0 or less, 70.0 or less, 60.0 or less, 50.0 or less, 40.0 or less, 30.0 or less, 20.0 or less, 15.0 or less, 10.0 or less, 8.00 or less, 5.00 or less, 2.50 or less, 2.00 or less, or 1.80 or less. From the viewpoint of achieving both the color development and the transfer resistance, the mass ratio (A)/(B) of the content of component (A) to the content of the component (B) may be 0.50 to 80.0, 0.80 to 20.0, 1.00 to 10.0, 1.10 to 5.00, 1.20 to 2.50, 1.30 to 2.00, or 1.40 to 1.80.

The temporary hair dye composition can obtain sufficient transfer resistance even if it does not comprise a coating agent; however, it may further comprise a coating agent. Examples of the coating agent include polyvinylpyrrolidone (PVP), polyvinyl alcohol, carboxyvinyl polymers or salts thereof, vinyl acetate/vinylpyrrolidone copolymers, hydroxypropyl methylcellulose, hydroxyethyl cellulose, carboxymethyl cellulose or salts thereof, polyacrylate, and acrylic copolymer emulsions.

The content of the coating agent may be 0.1 to 200 parts by mass, 0.1 to 50 parts by mass, or 1 to 30 parts by mass, based on 100 parts by mass of the component (A). In addition, the content of the coating agent may be 10 mass % or less, 5 mass % or less, 3 mass % or less, 1 mass % or less, or 0 mass % based on the total amount of the temporary hair dye composition.

The temporary hair dye composition may further comprise a surfactant from the viewpoint of obtaining favorable foaming. Examples of the surfactant include anionic surfactants, cationic surfactants, amphoteric surfactants, and nonionic surfactants. The nonionic surfactant can be used from the viewpoints of foam being easily adhered and color development.

Examples of the nonionic surfactant include polyoxyalkylene alkyl ether, glycerin alkyl ether, glycerin fatty acid ester, polyglycerin fatty acid ester, sorbitan fatty acid ester and alkylene glycol adducts thereof, polyalkylene glycol fatty acid ester, polyglycerin-modified silicone, and polyether-modified silicone. Examples of the anionic surfactant include alkyl phosphate, polyoxyalkylene alkyl ether phosphate, sulfonates, alkyl sulfate, polyaspartate, N-acyl methyl taurine salt, and polyoxyethylene alkyl ether sulfate. Examples of the cationic surfactant include alkylamine salts and alkyltrimethylammonium salts. Examples of the amphoteric surfactant include hydrogenated lecithin, carbobetaine type amphoteric surfactants, sulfobetaine type amphoteric surfactants, and amino acid type amphoteric surfactants. The surfactant may be used alone, or two or more thereof may be used in combination.

The content of the nonionic surfactant may be 0.01 to 10 mass % or 0.1 to 8 mass % based on the total amount of the temporary hair dye composition.

From the viewpoint of affinity with the soap base, a nonionic surfactant having an HLB value of 8.0 or more can be used. The HLB (hydrophilic-lipophilic balance) is calculated by the following formula according to the Griffin method:

HLB value=20×[(sum of molecular weights of hydrophilic moieties)/(total molecular weight)].

The temporary hair dye composition may further comprise a polyhydric alcohol from the viewpoint of adjusting the feel during use.

Examples of the polyhydric alcohol include 1,3-butylene glycol, glycerin, propanediol, dipropylene glycol, polyethylene glycol, pentanediol, hexanediol, pentylene glycol, caprylyl glycol, and ethylhexylglycerin. The polyhydric alcohol may be used alone, or two or more thereof may be used in combination. The number of hydroxy groups of the polyhydric alcohol may be 2 or more and may be 4 or less or 3 or less. The content of the polyhydric alcohol may be 1% by mass or more, 3% by mass or more, 5% by mass or more, or 8% by mass or more, and may be 20% by mass or less, 15% by mass or less, 12% by mass or less, or 10% by mass or less, or 1 to 15% by mass or 3 to 12% by mass, based on the total amount of the temporary hair dye composition.

Other Components

The temporary hair dye composition may comprise components other than the above-mentioned components. Blending can appropriately be done with the other components, including oil components, preservatives, pH adjusting agents, antioxidants, UV absorbing agents, chemicals, fragrances, powders and salts other than the component (B), which are commonly used in hair cosmetics. The total content of the other components may be, for example, 0 to 15 mass %, 1 to 12 mass %, 2 to 10 mass %, or 3 to 8 mass % based on the total amount of the temporary hair dye composition.

From the viewpoint of obtaining favorable foaming, the water content of the temporary hair dye composition may be 20 mass % or less, 15 mass % or less, 10 mass % or less, 8 mass % or less, or 6 mass % or less, or 0.1 mass % or more, 0.5 mass % or more, 1 mass % or more, 2 mass % or more, 3 mass % or more, or 4 mass % or more. The water content of the temporary hair dye composition can be measured by the Karl Fischer method using a Karl Fischer moisture meter (MKS-510N, commercially available from Kyoto Electronics Manufacturing Co., Ltd.).

In the temporary hair dye composition, water volatilizes depending on the storage environment of the composition, and its content may be less than a water content measured immediately after production or within a predetermined lapse of time (for example, within 24 hours), even in which case the effects of the temporary hair dye composition can be exhibited.

The temporary hair dye composition can be used in various dosage forms, and may be, for example, in the form of a liquid, gel, semi-solid (paste), solid, or powder. The liquid hair dye composition may be a temporary hair dye composition comprising a liquid soap or a temporary hair dye composition which is a water dispersion.

The forms of the solid temporary hair dye composition include, for examples, granules, sheets, and molded articles. The molded article may be a compression-molded article, an extruded molded article, a filled molded article, or a melt molded article.

The shape of the solid temporary hair dye composition may be a circular shape, an elliptical shape, a polygonal shape, a rectangular shape, a star shape, a heart shape, a flower shape or the like.

When the temporary hair dye composition is a molded article, the hardness measured with a rheometer at 25° C. may be 0.1 N or more or 1 to 15 N. The measurement conditions involve a bar shape with a diameter of 1 mm as a tip shape of the pressure-sensitive shaft, a penetration speed of 6 cm/min, and a penetration depth of 2 mm.

The temporary hair dye composition can be used by being mixed with water. For example, the temporary hair dye composition can be mixed with water, and the whipped foam can be applied to the hair. From the viewpoints of foaming and usability, the temporary hair dye composition may have a water content within the above-mentioned range, and may be in the form of a solid or powder, or may be in the form of a granule, sheet, or molded article. From the viewpoint of color development, 5 parts by mass or more, 10 to 70 parts by mass, or 20 to 60 parts by mass of water may be mixed to 100 parts by mass of the temporary hair dye composition.

Examples of the foaming tool include brushes, sponges, combs, screw brushes, and spatulas.

The temporary hair dye composition can be used for temporary dyeing of hair, eyebrows or the like, and may also have functions such as hair dressing, treatment, and eyebrow styling.

The temporary hair dye composition can be produced by mixing (A) the soap base and (B) the pigment and, in some examples, one or more other components.

(A) the soap base, (B) the pigment and, in some examples, one or more other components, can be uniformly mixed using three rollers, a super mixer, a Henschel mixer, an atomizer, a kneader or the like to obtain a base material of the temporary hair dye composition. After (A) the soap base, (B) the pigment, and any other components are mixed, the mixture may be heat-treated at 40 to 90° C.

Powders

The base material obtained above can be formed into powders using a mixer or the like according to a crushing method or a spray drying method to produce powders of the temporary hair dye composition. The water content in the powders may be within the above range.

Molded Articles

The base material obtained above can be filled into a predetermined container and compressed or heated/melted to produce a molded article of the temporary hair dye composition having a predetermined shape (for example, a compression-molded article, a melt molded article, or an extruded molded article). Alternatively, the base material obtained above can be heated/melted, filled into a predetermined container, and solidified to produce a molded article of the temporary hair dye composition having a predetermined shape (for example, a filled molded article). The molded article may have a water content within the above-mentioned range and a hardness within the above-mentioned range.

Granules

The base material obtained above can be formed into granules by a dry granulation or wet granulation method to produce granules of the temporary hair dye composition. The water content in the granule may be within the above-mentioned range.

Sheets

The base material obtained above can be formed into a sheet by a stretching method or a casting method to produce a sheet of the temporary hair dye composition. The water content in the sheet may be within the above-mentioned range.

The temporary hair dye compositions obtained by the above methods may be additionally dried. Drying may be performed in order to adjust the water content of the composition. Natural drying may be performed, or heating may be performed at 40 to 90° C. Drying may be performed so that the water content of the compositions are within the above-mentioned range.

Hereinafter, details of additional example temporary hair dye compositions will be described with reference to comparative examples.

Preparation of Temporary Hair Dye Compositions

### Examples 1 to 53 and Comparative Examples 1 and 2

According to formulations shown in Tables 1 to 9 temporary hair dye compositions were prepared by the following production method. Here, the numerical value of the amount added indicates the content (mass %) based on the total amount of the hair dye composition.

Details of raw materials in Tables 1 to 9 will be described below.

Component (A): Soap Base

Soap base-1: “Palmsabun PS-2405” (commercially available from IOI OLEOCHEMICAL, product name); a water content of 14 to 16%; a fatty acid salt content of 80 to 82%.

Production Method

Respective components in the table were uniformly dispersed using three rollers and uniformly mixed using a super mixer to obtain a base material of the temporary hair dye. The obtained base material was filled into a metal plate with a diameter of 43 mm, and compressed/molded so that the thickness after pressing was 3.5 mm, to form a molded article of the temporary hair dye. In Example 25, a molded article of the temporary hair dye was formed and then dried by heating at 60° C. for 12 hours.

The obtained molded articles were evaluated for respective items according to the evaluation methods described below.

Water Content

The molded article, 0.3 g, was used as a sample, and its water content was measured using a Karl Fischer moisture meter (MKS-510N, commercially available from Kyoto Electronics Manufacturing Co., Ltd.) by the Karl Fischer method. The water content was measured within 24 hours after the molded article was produced.

Evaluation 1

Water, 2 mL, was dropped onto each molded article so that its entire surface became wet, and foaming was performed with a face cleansing brush. Foams generated with the face cleansing brush were applied to goat hair bundles and dried to obtain colored hair bundles.

Transfer Resistance

Each of the obtained colored hair bundles was inserted between pieces of kitchen paper folded in half, and a weight was placed on the entire goat hair bundle to be pressed. The weight was a 1,000 mL-glass beaker having an outer diameter of 110 mm and the total height of 150 mm, which was adjusted to a total amount of 1,200 g by inclusion of water. After the sample was allowed to stand for 1 minute, the degree of adhesion of cosmetics was checked by visual observation, and evaluated according to the following determination criteria.

### Determination Criteria

A+: adhesion of cosmetics was hardly observed.
A: slight adhesion of cosmetics was observed.
B: adhesion of cosmetics was observed.
C: significant adhesion of cosmetics was observed.

Absence of Coarse Hair

For each of the obtained colored hair bundles, five panelists evaluated the absence of coarse hair (softness) when they touched the colored hair bundle with their fingers.

A three-step evaluation was performed according to the following evaluation criteria: a score was assigned to each sample, and, an average of the scores of all panelists was determined according to the following determination criteria.

### Score: Evaluation Criteria

3 points: softness without hair coarseness was felt relative to the comparative colored hair bundles prepared by the method described below.
2 points: softness with less hair coarseness was felt relative to the comparative colored hair bundles.
1 point: no significant difference in softness was felt relative to the comparative colored hair bundles.

### Determination Criteria (Average of Scores)

A+: 2.5 points or more
A: 2.0 points or more and less than 2.5 points
B: 1.5 points or more and less than 2.0 points
C: less than 1.5 points

Preparation of Comparative Colored Hair Bundle

A pigment, an oil component, and an organic modified clay mineral according to the comparative formulation described below were mixed and dispersed with rollers, and ethanol was then added thereto. The mixture was uniformly dispersed with a Disper to obtain a hair dye. A 0.2 g sample of the obtained hair dye was applied with a brush to 5 cm from the tip of a goat hair bundle, and dried at room temperature (25±1° C.) for 15 minutes, to obtain a comparative colored hair bundle.

Comparative Formulation

| | formulation proportion |
|---|---|
| Components added | (mass %) |

| | | |
|---|---|---|
| (1) | carboxybetaine resin-1 | 9.0 |
| (2) | ethanol | 55.0 |
| (3) | black iron oxide | 3.5 |
| (4) | red No. 202 | 0.8 |
| (5) | barium sulfate | 1.2 |
| (6) | yellow No. 4 aluminum lake | 6.5 |
| (7) | diisostearyl malate | 5.0 |
| (8) | sorbitan sesquiisostearate | 3.0 |
| (9) | cyclomethicone | 11.0 |
| (10) | stearalkonium bentonite | 5.0 |

carboxybetaine resin-1: “YUKAFORMER 202” (product name, commercially available from Mitsubishi Chemical Corporation)

Evaluation 2

### (Color Development)

Water, 2 mL, was dropped onto each molded article so that its entire surface became wet, and foaming was performed with a face cleansing brush. The whipped foam was applied to the subject's hair (black hair) and dried. Five panelists were asked to evaluate an impression of coloring on the hair according to the following determination criteria. Evaluation was performed according to the following criteria based on the total score of evaluations by the five panelists.

### [Determination Criteria]

3 points: good impression of coloring
2 points: weak impression of coloring
1 point: no impression of coloring

### [Determination Criteria (Average of Scores)]

A+: 2.5 points or more
A: 2.0 points or more and less than 2.5 points
B: 1.5 points or more and less than 2.0 points
C: less than 1.5 points

Evaluation 3

Water, 2 mL, was dropped onto each molded article so that its entire surface became wet, and foaming was performed with a face cleansing brush. The degree of foaming and the foam quality were evaluated according to the following determination criteria.

Foaming

### [Determination Criteria]

A: quick foaming
B: slightly slow foaming
C: slow foaming

Foam Quality (Fineness of Foam)

### [Determination Criteria]

A: fine foams
C: large foams mixed

TABLE 1

| | | Example | Example | Example | Example | Example | Example | Example |
|---|---|---|---|---|---|---|---|---|
| | | 1 | 2 | 3 | 4 | 5 | 6 | 7 |
| Soap base (A) | soap base-1 | 50 | 50 | 50 | 50 | 50 | 50 | 50 |
| Color pigment (B1) | organic pigment Red 226 | 5.5 | 5.5 | 5.5 | 5.5 | 5.5 | 5.5 | 5.5 |
| | aluminum powder pearl | — | — | 10 | — | — | — | — |
| | particle size of 20 μm | | | | | | | |
| | synthetic phlogopite base pearl | — | — | — | 10 | — | — | — |
| | particle size of 95 μm | | | | | | | |
| | mica based pearl | — | — | — | — | 10 | — | — |
| | particle size of 7 μm | | | | | | | |
| | glass based glitter | — | — | — | — | — | 10 | — |
| | particle size of 100 μm | | | | | | | |
| | glass based glitter | — | — | — | — | — | — | 10 |
| | particle size of 120 μm | | | | | | | |
| Extender pigment | dimethicone-treated synthetic | 25.5 | 24.9 | 15.5 | 15.5 | 15.5 | 15.5 | 15.5 |
| (B2) | phlogopite | | | | | | | |
| Polyhydric alcohol | 1,3-butylene glycol | 5 | 5 | 5 | 5 | 5 | 5 | 5 |
| | pentylene glycol | 5 | 5 | 5 | 5 | 5 | 5 | 5 |
| Surfactant | polyglyceryl-10 myristate | 3 | 3 | 3 | 3 | 3 | 3 | 3 |
| pH adjusting agent | citric acid | 6 | 6 | 6 | 6 | 6 | 6 | 6 |
| Preservative | phenoxyethanol | — | 0.6 | — | — | — | — | — |
| Water content of molded article (mass %) | | 5 | 5 | 5 | 5 | 5 | 5 | 5 |
| Evaluation 1 | transfer resistance | A+ | A+ | A+ | A+ | A+ | A+ | A+ |
| | absence of coarse hair | A+ | A+ | A+ | A+ | A+ | A+ | A+ |
| Evaluation 2 | good color development | A+ | A+ | A+ | A+ | A+ | A+ | A+ |
| Evaluation 3 | foaming | A | A | A | A | A | A | A |
| | foam quality (fineness of foam) | A | A | A | A | A | A | A |

TABLE 2

| | | Example | Example | Example | Example | Example | Example | Example |
|---|---|---|---|---|---|---|---|---|
| | | 8 | 9 | 10 | 11 | 12 | 13 | 14 |
| Soap base (A) | soap base-1 | 50 | 50 | 50 | 50 | 50 | 50 | 50 |
| Color pigment (B1) | organic pigment Red 226 | 5.5 | 5.5 | 5.5 | 5.5 | 5.5 | 5.5 | 5.5 |
| Extender pigment | mica | 25.5 | — | — | — | — | — | — |
| (B2) | synthetic phlogopite | — | 25.5 | — | — | — | — | — |
| | kaolin | — | — | 25.5 | — | — | — | — |
| | talc | — | — | — | 25.5 | — | — | — |
| | dimethicone-treated mica | — | — | — | — | 25.5 | — | — |
| | dimethicone-treated talc | — | — | — | — | — | 25.5 | — |
| | dimethicone-treated silica | — | — | — | — | — | — | 25.5 |
| Polyhydric alcohol | 1,3-butylene glycol | 5 | 5 | 5 | 5 | 5 | 5 | 5 |
| | pentylene glycol | 5 | 5 | 5 | 5 | 5 | 5 | 5 |
| Surfactant | polyglyceryl-10 myristate | 3 | 3 | 3 | 3 | 3 | 3 | 3 |
| pH adjusting agent | citric acid | 6 | 6 | 6 | 6 | 6 | 6 | 6 |
| Water content of molded article (mass %) | | 5 | 5 | 5 | 5 | 5 | 5 | 5 |
| Evaluation 1 | transfer resistance | A+ | A+ | A+ | A+ | A+ | A+ | A+ |
| | absence of coarse hair | A+ | A+ | A+ | A+ | A+ | A+ | A+ |
| Evaluation 2 | good color development | A+ | A+ | A+ | A+ | A+ | A+ | A+ |
| Evaluation 3 | foaming | A | A | A | A | A | A | A |
| | foam quality (fineness of | A | A | A | A | A | A | A |
| | foam) | | | | | | | |

TABLE 3

| | | Example | Example | Example | Example | Example |
|---|---|---|---|---|---|---|
| | | 15 | 16 | 17 | 18 | 19 |
| Soap base (A) | soap base-1 | 50 | 55 | 50 | 50 | 50 |
| Color pigment (B1) | organic pigment Red 226 | 15 | 40 | 35 | 0.5 | 1 |
| Extender pigment | dimethicone-treated synthetic | 25 | 5 | 5 | 35.4 | 30 |
| (B2) | phlogopite | | | | | |
| Polyhydric alcohol | 1,3-butylene glycol | 10 | — | 4 | 5 | 5 |
| | pentylene glycol | — | — | 4 | 3 | 5 |
| Surfactant | polyglyceryl-10 myristate | — | — | — | 6 | 3 |
| pH adjusting agent | citric acid | — | — | 2 | 0.1 | 6 |
| Water content of molded article (mass %) | | 5 | 5 | 5 | 5 | 5 |
| Evaluation 1 | transfer resistance | A+ | B | A | A+ | A+ |
| | absence of coarse hair | A+ | A | A | A+ | A+ |
| Evaluation 2 | good color development | A+ | A+ | A+ | B | A |
| Evaluation 3 | foaming | A | A | A | A | A |
| | foam quality (fineness of foam) | A | A | A | A | A |

TABLE 4

| | | Example | Example | Example | Example | Example | Example |
|---|---|---|---|---|---|---|---|
| | | 20 | 21 | 22 | 23 | 24 | 25 |
| Soap base (A) | soap base-1 | 90 | 33 | 77 | 41.4 | 45 | 45 |
| Color pigment (B1) | organic pigment Red 226 | 5 | 5.5 | 3 | — | — | — |
| | dimethicone-treated yellow iron | — | — | — | 10.5 | — | — |
| | oxide | | | | | | |
| | dimethicone-treated ultramarine | — | — | — | — | 10.5 | 10.5 |
| Extender pigment | dimethicone-treated synthetic | 5 | 54.5 | 3 | 29.1 | 25.5 | 25.5 |
| (B2) | phlogopite | | | | | | |
| Polyhydric alcohol | 1,3-butylene glycol | — | 5 | 5 | 5 | 5 | 5 |
| | pentylene glycol | — | 2 | 5 | 5 | 5 | 5 |
| Surfactant | polyglyceryl-10 myristate | — | — | 1 | 3 | 3 | 3 |
| Coating agent | PVP | — | — | — | — | — | — |
| pH adjusting agent | citric acid | — | — | 6 | 6 | 6 | 6 |
| Water content of molded article (mass %) | | 9 | 3 | 8 | 3.5 | 4 | 0.3 |
| Evaluation 1 | transfer resistance | A+ | B | A+ | A+ | A+ | A+ |
| | absence of coarse hair | B | A | B | A+ | A+ | A+ |
| Evaluation 2 | good color development | A+ | A+ | A+ | A+ | A+ | A+ |
| Evaluation 3 | foaming | A | B | A | A | A | A |
| | foam quality (fineness of foam) | A | A | A | A | A | A |

TABLE 5

| | | Example | Example | Example | Example | Example | Example | Example |
|---|---|---|---|---|---|---|---|---|
| | | 26 | 27 | 28 | 29 | 30 | 31 | 32 |
| Soap base (A) | soap base-1 | 80 | 77 | 77 | 77 | 72.9 | 70 | 70 |
| Color pigment (B1) | organic pigment Red 226 | 0.5 | 1 | 0.5 | 3 | 4.05 | 10.5 | 0.5 |
| Extender pigment | dimethicone-treated | 0.5 | 3 | 3.5 | 1 | 4.05 | 0.5 | 10.5 |
| (B2) | synthetic phlogopite | | | | | | | |
| Polyhydric alcohol | 1,3-butylene glycol | 5 | 5 | 5 | 5 | 5 | 5 | 5 |
| | pentylene glycol | 5 | 5 | 5 | 5 | 5 | 5 | 5 |
| Surfactant | polyglyceryl-10 myristate | 3 | 3 | 3 | 3 | 3 | 3 | 3 |
| pH adjusting agent | citric acid | 6 | 6 | 6 | 6 | 6 | 6 | 6 |
| Water content of molded article (mass %) | | 7.5 | 8 | 8 | 8 | 7 | 7 | 7 |
| Evaluation 1 | transfer resistance | A+ | A+ | A+ | A+ | A+ | A+ | A+ |
| | absence of coarse hair | A | A | A | A | A | A | A |
| Evaluation 2 | good color development | B | A | B | A+ | A+ | A+ | B |
| Evaluation 3 | foaming | A | A | A | A | A | A | A |
| | foam quality (fineness of | A | A | A | A | A | A | A |
| | foam) | | | | | | | |

TABLE 6

| | | Example | Example | Example | Example | Example | Example |
|---|---|---|---|---|---|---|---|
| | | 33 | 34 | 35 | 36 | 37 | 38 |
| Soap base (A) | soap base-1 | 61 | 58 | 58 | 55 | 47.1 | 45 |
| Color pigment (B1) | organic pigment Red 226 | 10 | 21.5 | 1.5 | 19 | 33.4 | 4.5 |
| Extender pigment | dimethic one-treated synthetic | 10 | 1.5 | 21.5 | 7 | 0.5 | 31.5 |
| (B2) | phlogopite | | | | | | |
| Polyhydric alcohol | 1,3-butylene glycol | 5 | 5 | 5 | 5 | 5 | 5 |
| | pentylene glycol | 5 | 5 | 5 | 5 | 5 | 5 |
| Surfactant | polyglyceryl-10 myristate | 3 | 3 | 3 | 3 | 3 | 3 |
| pH adjusting agent | citric acid | 6 | 6 | 6 | 6 | 6 | 6 |
| Water content of molded article (mass %) | | 6 | 6 | 6 | 5.5 | 4.5 | 4.5 |
| Evaluation 1 | transfer resistance | A | A | A+ | A | A | A+ |
| | absence of coarse hair | A | A+ | A+ | A+ | A+ | A+ |
| Evaluation 2 | good color development | A+ | A+ | A+ | A+ | A+ | A+ |
| Evaluation 3 | foaming | A | A | A | A | A | A |
| | foam quality (fineness of foam) | A | A | A | A | A | A |

TABLE 7

| | | Example | Example | Example | Example | Example | Example | Example | Example |
|---|---|---|---|---|---|---|---|---|---|
| | | 39 | 40 | 41 | 42 | 43 | 44 | 45 | 46 |
| Soap base (A) | soap base-1 | 45 | 45 | 45 | 42.6 | 41.4 | 40 | 40 | 40 |
| Color pigment (B1) | organic pigment Red 226 | 22.5 | 25.5 | 20.5 | 4.3 | 29.1 | 25.5 | 1 | 40.5 |
| Extender | dimethicone-treated | 13.5 | 10.5 | 15.5 | 34.1 | 10.5 | 15.5 | 40 | 0.5 |
| pigment (B2) | synthetic phlogopite | | | | | | | | |
| Polyhydric alcohol | 1,3-butylene glycol | 5 | 5 | 5 | 5 | 5 | 5 | 5 | 5 |
| | pentylene glycol | 5 | 5 | 5 | 5 | 5 | 5 | 5 | 5 |
| Surfactant | polyglyceryl-10 | 3 | 3 | 3 | 3 | 3 | 3 | 3 | 3 |
| | myristate | | | | | | | | |
| pH adjusting agent | citric acid | 6 | 6 | 6 | 6 | 6 | 6 | 6 | 6 |
| Water content of molded article (mass %) | | 4.5 | 4.5 | 4.5 | 4 | 4 | 4 | 3.5 | 3.5 |
| Evaluation 1 | transfer resistance | A | A | A | A+ | A | A | A | A |
| | absence of coarse hair | A+ | A+ | A+ | A+ | A+ | A | A | A |
| Evaluation 2 | good color development | A+ | A+ | A+ | A+ | A+ | A+ | A | A+ |
| Evaluation 3 | foaming | A | A | A | A | A | A | A | A |
| | foam quality (fineness | A | A | A | A | A | A | A | A |
| | of foam) | | | | | | | | |

TABLE 8

| | | Example | Example | Example | Example | Example | Example | Example |
|---|---|---|---|---|---|---|---|---|
| | | 47 | 48 | 49 | 50 | 51 | 52 | 53 |
| Soap base (A) | soap base-1 | 36 | 36 | 33 | 30 | 30 | 30 | 30 |
| Color pigment (B1) | organic pigment Red 226 | 44 | 1 | 1 | 15 | 46.2 | 26.5 | 20 |
| Extender | dimethicone-treated | 1 | 44 | 47 | 36 | 4.8 | 24.5 | 31 |
| pigment (B2) | synthetic phlogopite | | | | | | | |
| Polyhydric alcohol | 1,3-butylene glycol | 5 | 5 | 5 | 5 | 5 | 5 | 5 |
| | pentylene glycol | 5 | 5 | 5 | 5 | 5 | 5 | 5 |
| Surfactant | polyglyceryl-10 myristate | 3 | 3 | 3 | 3 | 3 | 3 | 3 |
| pH adjusting agent | citric acid | 6 | 6 | 6 | 6 | 6 | 6 | 6 |
| Water content of molded article (mass %) | | 2.5 | 2.5 | 2.5 | 3 | 3 | 3 | 3 |
| Evaluation 1 | transfer resistance | B | A | B | B | B | B | B |
| | absence of coarse hair | A | A | A | A | A | A | A |
| Evaluation 2 | good color development | A+ | A | A | A+ | A+ | A+ | A+ |
| Evaluation 3 | foaming | A | A | A | B | B | B | B |
| | foam quality (fineness of | A | A | A | A | B | A | A |
| | foam) | | | | | | | |

TABLE 9

| | | Comparative | Comparative |
|---|---|---|---|
| | | Example 1 | Example 2 |
| Soap base (A) | soap base-1 | 0 | 0 |
| Color | organic pigment Red 226 | 5.5 | 5.5 |
| pigment (B1) | dimethicone-treated | — | — |
| | yellow iron oxide | | |
| | dimethicone-treated | — | — |
| | ultramarine | | |
| Extender | dimethicone-treated | 75.5 | 71.5 |
| pigment (B2) | synthetic phlogopite | | |
| Polyhydric | 1,3-butylene glycol | 5 | 5 |
| alcohol | pentylene glycol | 5 | 5 |
| Surfactant | polyglyceryl-10 myristate | 3 | 3 |
| Coating agent | PVP | — | 10 |
| pH adjusting | citric acid | 6 | — |
| agent | | | |
| Water content of molded article (mass %) | | 0 | 0 |
| Evaluation 1 | transfer resistance | C | A |
| | absence of coarse hair | A | C |
| Evaluation 2 | good color development | A | B |
| Evaluation 3 | foaming | C | C |
| | foam quality (fineness of | C | C |
| | foam) | | |

### Example 54: Temporary Hair Dye for Eyebrows

| | | formulation proportion |
|---|---|---|
| Components | | (mass %) |
| 1. | dimethicone-treated synthetic phlogopite | 32.2 |
| 2. | red oxide | 4.6 |
| 3. | yellow iron oxide | 1.7 |
| 4. | carbon black | 0.2 |
| 5. | titanium oxide | 1.3 |
| 6. | soap base-1 | 50.0 |
| 7. | 1,3-butylene glycol | 5.0 |
| 8. | pentylene glycol | 5.0 |

Details of soap base-1 (component 6) are described above.

Production Method

Components 1 to 8 were uniformly dispersed using three rollers and mixed using a super mixer to obtain a base material of the hair dye composition. The obtained base material was filled into a metal plate with a diameter of 43 mm and compressed/molded so that the thickness after pressing was 3.5 mm, to form a molded article of the temporary hair dye for eyebrows.

Evaluation

When the obtained temporary hair dye for eyebrows was evaluated in the same manner as above, it was evaluated as “A+” for the transfer resistance, “A+” for the coarseness of hair, “A+” for the good color development, “A” for the foaming, and “A” for the foam quality.

The water content of the molded article was 0.3 mass %.

### Example 55: Temporary Hair Dye for Eyebrows

| | | formulation proportion |
|---|---|---|
| Components | | (mass %) |
| 1. | dimethicone-treated synthetic phlogopite | 24.4 |
| 2. | red oxide | 9.2 |
| 3. | yellow iron oxide | 3.4 |
| 4. | carbon black | 0.4 |
| 5. | titanium oxide | 2.6 |
| 6. | soap base-1 | 50.0 |
| 7. | 1,3-butylene glycol | 5.0 |
| 8. | pentylene glycol | 5.0 |

Details of soap base-1 (component 6) are described above.

Production Method

Components 1 to 8 were uniformly dispersed using three rollers and mixed using a super mixer to obtain a base material of the hair dye composition. The obtained base material was filled into a metal plate with a diameter of 43 mm and compressed/molded so that the thickness after pressing was 3.5 mm to form a molded article of the temporary hair dye for eyebrows.

Evaluation

When the obtained temporary hair dye for eyebrows was evaluated in the same manner as above, it was evaluated as “A” for the transfer resistance, “A” for the coarseness of hair, “A+” for the good color development, “A” for the foaming, and “A” for the foam quality. The water content of the molded article was 5 mass %.

### Example 56: Temporary Hair Dye

| | | formulation proportion |
|---|---|---|
| Components | | (mass %) |
| 1. | dimethicone-treated synthetic phlogopite | 25.4 |
| 2. | carbon black | 0.3 |
| 3. | dimethicone-treated yellow iron oxide | 1.2 |
| 4. | dimethicone-treated titanium oxide | 0.9 |
| 5. | dimethicone-treated red oxide | 3.2 |
| 6. | soap base-1 | 50.0 |
| 7. | 1,3-butylene glycol | 5.0 |
| 8. | pentylene glycol | 5.0 |
| 9. | sodium methyl cocoyl taurate | 3.0 |
| 10. | citric acid | 6.0 |

Details of soap base-1 (component 6) are described above.

Production Method

Components 1 to 10 were uniformly dispersed using three rollers and mixed using a super mixer to obtain a base material of the hair dye composition. The obtained base material was filled into a metal plate with a diameter of 43 mm and heated and then compressed/molded so that the thickness after pressing was 3.5 mm to form a molded article of the temporary hair dye.

Evaluation

When the obtained temporary hair dye was evaluated in the same manner as above, it was evaluated as “A+” for the transfer resistance, “A+” for the coarseness of hair, “A+” for the good color development, “A” for the foaming, and “A” for the foam quality. The water content of the molded article was 5 mass %.

### Example 57: Temporary Hair Dye

| | | formulation proportion |
|---|---|---|
| Components | | (mass %) |
| 1. | dimethicone-treated synthetic phlogopite | 9.5 |
| 2. | red 226 | 5.5 |
| 3. | soap base-2 | 80.0 |
| 4. | 1,3-butylene glycol | 2.5 |
| 5. | pentylene glycol | 2.5 |

Details of soap base-2 (Component 3) are as follows:

Soap base-2: “SOAPLEX HOSOVEG” (commercially available from Laboratoire Sandralex, product name); a water content of 7 to 13%; and a fatty acid salt content of 81 to 89%.

Production Method

Components 1 to 5 were uniformly dispersed using three rollers and mixed using a super mixer to obtain a base material of the hair dye composition. The obtained base material was filled into a metal plate with a diameter of 43 mm and compressed/molded so that the thickness after pressing was 3.5 mm to form a molded article of the temporary hair dye.

Evaluation

When the obtained temporary hair dye was evaluated in the same manner as above, it was evaluated as “A+” for the transfer resistance, “A” for the coarseness of hair, “A+” for the good color development, “A” for the foaming, and “A” for the foam quality. The water content of the molded article was 5 mass %.

### Example 58: Temporary Hair Dye (Water Dispersion)

| | | formulation proportion |
|---|---|---|
| Components | | (mass %) |
| 1. | dimethicone-treated synthetic phlogopite | 59.5 |
| 2. | red 226 | 5.5 |
| 3. | soap base-1 | 5.0 |
| 4. | polyvinylpyrrolidone (PVP) | 10.0 |
| 5. | water | 20.0 |

Details of soap base-1 (Component 3) are described above.

Production Method

After component 4 was dispersed in the water of Component 5, the obtained dispersion was mixed with Components 1 to 3, which were dissolved/heated, using a Disper to obtain a water dispersion of the temporary hair dye.

Evaluation

When the obtained temporary hair dye was evaluated in the same manner as above, it was evaluated as “B” for the transfer resistance, “B” for the coarseness of hair, and “A” for the good color development. Regarding the foaming and the foam quality, no sufficient foam was obtained.

### Example 59: Temporary Hair Dye (Liquid)

| | | formulation proportion |
|---|---|---|
| Components | | (mass %) |
| 1. | dimethicone-treated synthetic phlogopite | 8.5 |
| 2. | red 226 | 5.5 |
| 3. | soap base-3 | 70.0 |
| 4. | polyvinylpyrrolidone (PVP) | 10.0 |
| 5. | citric acid | 6.0 |

Details of soap base-3 (Component 3) are as follows:

Soap base-3: “ALFOAM K-100” (commercially available from New Japan Chemical Co., Ltd., product name); a water content of 67 to 73%; a fatty acid salt content of 27 to 33%.

Production Method

After component 4 was dispersed in the water of Component 5, the obtained dispersion was mixed with Components 1 to 3, which were dissolved/heated, using a Disper to obtain a liquid temporary hair dye.

Evaluation

When the obtained temporary hair dye was evaluated in the same manner as above, it was evaluated as “B” for the transfer resistance, “A” for the coarseness of hair, “B” for the good color development, “A” for the foaming, and “A” for the foam quality. The water content was 45%.

### Example 60: Temporary Hair Dye (Paste)

| | | formulation proportion |
|---|---|---|
| Components | | (mass %) |
| 1. | dimethicone-treated synthetic phlogopite | 3.0 |
| 2. | red 226 | 5.5 |
| 3. | soap base-1 | 50.0 |
| 4. | 1,3-butylene glycol | 20.0 |
| 5. | pentylene glycol | 20.0 |
| 6. | polyglyceryl-10 myristate | 1.0 |
| 7. | citric acid | 0.5 |

Details of soap base-1 (Component 3) are described above.

Production Method

Components 1 to 7 were mixed using three rollers to obtain a paste temporary hair dye.

Evaluation

When the obtained temporary hair dye (paste) was evaluated in the same manner as above, it was evaluated as “B” for the transfer resistance, “B” for the coarseness of hair, “A” for the good color development, “A” for the foaming, and “A” for the foam quality. The water content of the paste was 5 mass %.

### Example 61: Temporary Hair Dye

| Components | | formulation proportion (mass %) |
|---|---|---|
| 1. | dimethicone-treated mica base | 25.5 |
| | pearl particle size 20 μm | |
| 2. | red 226 | 5.5 |
| 3. | soap base-1 | 50.0 |
| 4. | 1,3-butylene glycol | 5.0 |
| 5. | pentylene glycol | 5.0 |
| 6. | polyglyceryl-10 myristate | 3.0 |
| 7. | citric acid | 6.0 |

Details of soap base-1 (Component 3) are described above.

Production Method

Components 1 to 7 were uniformly dispersed using three rollers and mixed using a super mixer to obtain a base material of the hair dye composition. The obtained base material was filled into a metal plate with a diameter of 43 mm and compressed/molded so that the thickness after pressing was 3.5 mm, to form a molded article of the temporary hair dye.

Evaluation

When the obtained temporary hair dye was evaluated in the same manner as above, it was evaluated as “B” for the transfer resistance, “B” for the coarseness of hair, “A+” for the good color development, “A” for the foaming, and “A” for the foam quality. The water content of the molded article was 5 mass %.

### Example 62: Temporary Hair Dye (Elastic Solid Form)

| | | formulation proportion |
|---|---|---|
| Components | | (mass %) |
| 1. | dimethicone-treated synthetic phlogopite | 20.5 |
| 2. | red 226 | 5.5 |
| 3. | soap base-1 | 50.0 |
| 4. | 1,3-butylene glycol | 5.0 |
| 5. | pentylene glycol | 5.0 |
| 6. | polyglyceryl-10 myristate | 3.0 |
| 7. | citric acid | 6.0 |
| 8. | xanthan gum | 5.0 |

Details of soap base-1 (Component 3) are described above.

Production Method

Components 1 to 7 were mixed using three rollers to obtain a base material of the hair dye composition. The obtained base material was filled into a metal plate with a diameter of 43 mm and dried so that the thickness after pressing was 3.5 mm, to form an elastic solid temporary hair dye.

Evaluation

When the obtained temporary hair dye was evaluated in the same manner as above, it was evaluated as “B” for the transfer resistance, “B” for the coarseness of hair, “A” for the good color development, “A” for the foaming, and “A” for the foam quality. The water content of the temporary hair dye was 5 mass %.

What is claimed is:

1. A temporary hair dye composition comprising
(A) a soap base, and
(B) a pigment.

2. The temporary hair dye composition according to claim 1, wherein the temporary hair dye composition has a water content of 20% or less as measured by a Karl Fischer method.

3. The temporary hair dye composition according to claim 1, wherein the temporary hair dye composition is in the form of a powder or solid.

4. The temporary hair dye composition according to claim 1, wherein the content of the soap base, based on the total amount of the temporary hair dye composition, is 30 to 90 mass %.

5. The temporary hair dye composition according to claim 1, wherein the mass ratio (A)/(B) of the content of the soap base to the content of the pigment is 0.5 to 80.

6. The temporary hair dye composition according to claim 1,
wherein the pigment comprises a color pigment and an extender pigment,
wherein the content of the color pigment is 0.5 to 50 parts by mass, and
wherein the content of the extender pigment is 0.5 to 50 parts by mass, based on the total content of 100 parts by mass of the soap base, the color pigment and the extender pigment.

7. The temporary hair dye composition according to claim 6, wherein the total content of the color pigment and the extender pigment is 1 to 55 parts by mass.

8. The temporary hair dye composition according to claim 1, further comprising a nonionic surfactant.

9. The temporary hair dye composition according to claim 1, wherein the temporary hair dye composition is used by being mixed with water.

10. The temporary hair dye composition according to claim 1,
wherein the soap base comprises a fatty acid salt, and
wherein the content of the fatty acid salt is 60 to 95 mass % based on the total amount of the soap base.

11. The temporary hair dye composition according to claim 1, wherein the content of the pigment is 1 to 60 mass % based on the total amount of the temporary hair dye composition.

12. The temporary hair dye composition according to claim 1, wherein the mass ratio (A)/(B) of the content of the soap base to the content of the pigment is 1.00 to 10.0.

13. The temporary hair dye composition according to claim 1,
wherein the pigment comprises a color pigment and an extender pigment,
wherein the content of the soap base is 40 to 60 mass %,
wherein the content of the color pigment is 1.5 to 20 mass %, and
wherein the content of the extender pigment is 10 to 35 mass %, based on the total amount of the temporary hair dye composition.

14. The temporary hair dye composition according to claim 1,
wherein the temporary hair dye composition further comprises a coating agent, and
wherein the content of the coating agent is 5 mass % or less based on the total amount of the temporary hair dye composition.

15. The temporary hair dye composition according to claim 1, further comprising a polyhydric alcohol.

16. A method for temporarily coloring hair comprising:
mixing the temporary hair dye composition according to claim 1 with water to generate a foam; and
applying the foam to the hair.
